(19) United States
(12) Patent Application Publication
(10) Pub. No.: US 2021/0292404 A1
(43) Pub. Date: Sep. 23, 2021

(54) USE OF IL-1BETA BINDING ANTIBODY CANAKINUMAB FOR TREATING OR ALLEVATING SYMPTOMS PULMONARY SARCOIDOSIS

(71) Applicant: NOVARTIS AG, Basel (CH)

(72) Inventor: Stephen John Oliver, Basel (CH)

(21) Appl. No.: 16/319,630

(22) PCT Filed: Jul. 19, 2017
(86) PCT No.: PCT/IB2017/054360

§ 371 Date: Jan. 22, 2019

Related U.S. Application Data
(60) Provisional application No. 62/365,017, filed on Jul. 21, 2016.

(51) Int. Cl. C07K 16/24; A61K 45/06; A61M 5/20; A61K 9/00

(52) U.S. Cl. CPC C07K 16/245; A61K 45/06; A61K 2039/545; A61K 9/0019; A61M 5/20

(57) ABSTRACT

The present invention relates to a method for treating or alleviating the symptoms of pulmonary sarcoidosis in a subject, comprising administering about 25 mg to about 300 mg of canakinumab.

### CROSS REFERENCE TO RELATED APPLICATIONS

This application is a U.S. National Phase Application under 35 U.S.C. § 371 of International Application PCT/IB2017/054360, filed on Jul. 19, 2017, which claims priority to U.S. Provisional Application No. 62/365,017, filed on Jul. 21, 2016, the contents of which are hereby incorporated by reference in their entireties.

### TECHNICAL FIELD

The present invention relates to a novel use and dosage regimen of canakinumab for treating or alleviating the symptoms of pulmonary sarcoidosis.

### BACKGROUND OF THE INVENTION

Chronic sarcoidosis is a systemic disease characterized by development of granulomas, inflammation and accompanying fibrotic tissue reactions (Chen and Moller 2011). Although any organ can be affected, most common disease manifestations are found in lung, skin, and eye tissues. Sarcoidosis can lead to ocular pain or loss of vision, skin lesions, congestive heart failure, cardiac arrhythmias, neurologic impairment, fatigue, depression, hypercalcemia, renal impairment and end organ failure.

IL-1β is a pro-inflammatory cytokine produced by a variety of cell types, particularly mononuclear phagocytes, in response to injury, infection and inflammation. In sarcoidosis, IL-1β has been shown to be an important contributor to maintaining macrophage and T cell alveolitis and epithelioid cell granuloma formation (Hunninghake 1984). Epithelioid cells, the predominant cell type within sarcoid granulomas, have been shown to strongly express IL-1β (Devergne et al. 1992). IL-1β is known to induce and enhance granuloma formation in vitro and in vivo (Kasahara et al. 1989, Terao et al. 1993). Thus, IL-1β represents a potential therapeutic target for sarcoidosis.

There are no approved therapies for sarcoidosis. Corticosteroid use has long comprised the standard of care, with diverse and unapproved, secondary immunosuppressive usage (e.g., methotrexate, azathioprine) as needed, all of which are accompanied by treatment-related serious adverse events (Paramothayan and Lasserson 2008, Baughman and Nunes 2012). Clinical trials in sarcoidosis with biological therapies targeting the adaptive immune response have not met expectations. The typical clinical course for pulmonary sarcoidosis patients is characterized by progressive and debilitating declines in lung function, with the primary causes of morbidity and mortality including pulmonary hypertension and fibrosis (Baughman and Lower 2011). Overall, the quality of life of patients with sarcoidosis is greatly diminished. Thus there is an unmet medical need in patients with sarcoidosis for a disease modifying agent that induces resolution of granulomas and prevents deterioration or improves lung function and restores quality of life.

### SUMMARY OF THE INVENTION

Accordingly, in one aspect, the present invention is directed to a method of treating or alleviating the symptoms of pulmonary sarcoidosis in a subject, comprising administering about 25 mg to about 300 mg of canakinumab.

In another aspect, the present invention is directed to canakinumab for use as a medicament for treating or alleviating the symptoms of pulmonary sarcoidosis in a subject, comprising administering about 25 mg to about 300 mg of canakinumab.

In yet another aspect, the present invention is directed to the use of canakinumab for the manufacture of a medicament for treating or alleviating the symptoms of pulmonary sarcoidosis in a subject, comprising administering about 25 mg to about 300 mg of canakinumab.

Further features and advantages of the invention will become apparent from the following description.

### BRIEF DESCRIPTION OF DRAWINGS

FIG. 1. Graphic representation of study design.

### DETAILED DESCRIPTION OF THE INVENTION

Sarcoidosis is an abnormal collection of inflammatory cells that form nodules known as granulomas. Any organ can be affected, but most commonly the disease manifests in the lungs as pulmonary sarcoidosis, and can lead to pulmonary hypertension.

The present invention provides, inter alia, a method of treating or alleviating the symptoms of pulmonary sarcoidosis in a subject, comprising administering about 25 mg to about 300 mg of canakinumab.

In one embodiment of any method of the invention the subject is exhibiting at least one of the following conditions before treatment: a) reduced lung function, b) dyspnea of at least 1 on the Modified Medical Research Council (MMRC) Dyspnea scale and c) abnormalities in the lung parenchyma. Lung function will improve after treatment with the methods and uses according to the present invention.

Canakinumab (ACZ885) is a fully human monoclonal anti-human IL-1β antibody of the IgG1/k isotype, being developed for the treatment of IL-1β driven inflammatory diseases. It is designed to bind to human IL-1β and thus blocks the interaction of this cytokine with its receptors. The antagonism of the IL-1β mediated inflammation using canakinumab in lowering C-reactive protein (CRP) and other inflammatory marker levels has shown an acute phase response in patients with Cryopyrin-Associated Periodic Syndrome (CAPS) and rheumatoid arthritis. Canakinumab reduces the risk of continued formation of granulomas in patients with pulmonary sarcoidosis by preventing IL-1β mediated inflammation and stopping and reversing the progression of the disease. Canakinumab is approved under the trade name Ilaris®. Canakinumab is disclosed in WO02/16436 which is hereby incorporated by reference in its entirety.

Lung (pulmonary) function can be measured using any known method, including, but not limited to, spirometry and plethysmography. Parameters of lung function include, but are not limited to, forced vital capacity (FVC), forced expiratory volume in one second (FEV1), forced expiratory volume in three seconds (FEV3), forced expiratory volume in six seconds (FEV6), mid-expiratory flow rate (the average flow from the point at which 25 percent of the FVC has been exhaled to the point at which 75 percent of the FVC has been exhaled: FEF25-75), FEV1/FVC, FEV3/FVC, FEV6/FVC and 1-(FEV3/FVC).

In different embodiments, the subject has reduced lung function before treatment. In some embodiments the subject has predicted forced vital capacity of ≤90%. In some embodiments the subject has predicted forced vital capacity of ≤85%. In some embodiments the subject has predicted forced vital capacity of ≤80%. In some embodiments the subject has predicted forced vital capacity of ≤75%. In some embodiments the subject has predicted forced vital capacity of ≤70%. In some embodiments the subject has predicted forced vital capacity of ≤65%. In some embodiments the subject has predicted forced vital capacity of ≤60%. In some embodiments the subject has predicted forced vital capacity of ≤55%. In some embodiments the subject has predicted forced vital capacity of ≤50%.

Herein, the forced vital capacity (FVC) is the maximal amount of air that the subject can forcibly exhale after taking a maximal inhalation. Predicted FVC is expressed as a percentage of the normal expected value, stratified by gender, age, height, and race (% FVC). An increase can be measured based on the predicted FVC based on a large patient population, on the FVC measured in a control population, or on the individual subject's predicted FVC prior to administration (baseline). In some embodiments, the methods described herein can increase the predicted FVC, as compared to the subject's baseline predicted FVC. In some embodiments, the increased predicted FVC is pre-bronchodilator FVC. In some embodiments, the increased predicted FVC is post-bronchodilator FVC. In some embodiments, the increased predicted FVC is pre-bronchodilator FVC and post-bronchodilator FVC.

In some embodiments, the methods and uses provided herein can increase predicted FVC pre-bronchodilator by at least 3% or at least 4% or at least 5% or at least 6% or at least 7% or at least 8% or at least 9% or at least 10%.

In some embodiments of the invention, predicted FVC pre-bronchodilator is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

In some embodiments, the methods and uses provided herein can increase predicted FVC post-bronchodilator by at least 3% or at least 4% or at least 5% or at least 6% or at least 7% or at least 8% or at least 9% or at least 10%.

In some embodiments of the invention, predicted FVC post-bronchodilator is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

In some embodiments, the methods and uses provided herein can increase predicted FVC pre- and post-bronchodilator by at least 3% or at least 4% or at least 5% or at least 6% or at least 7% or at least 8% or at least 9% or at least 10%.

In some embodiments of the invention, predicted FVC pre- and post-bronchodilator is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment. The methods and uses provided herein can increase absolute forced vital capacity (FVC) in subjects with pulmonary sarcoidosis. An increase can be measured based on the expected FVC based on a large patient population, on the FVC measured in a control population, or on the individual subject's FVC prior to administration (baseline). In some embodiments, the methods described herein can increase the absolute FVC, as compared to the subject's baseline FVC. In some embodiments, the increased absolute FVC is pre-bronchodilator FVC. In some embodiments, the increased absolute FVC is post-bronchodilator FVC. In some embodiments, the increased absolute FVC is pre-bronchodilator FVC and post-bronchodilator FVC.

In some embodiments, the methods and uses provided herein can increase pre-bronchodilator absolute FVC by at least 3% or at least 4% or at least 5% or at least 6% or at least 7% or at least 8% or at least 9% or at least 10%.

In some embodiments of the invention, pre-bronchodilator absolute FVC is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

In some embodiments, the methods and uses provided herein can increase post-bronchodilator absolute FVC by at least 3% or at least 4% or at least 5% or at least 6% or at least 7% or at least 8% or at least 9% or at least 10%.

In some embodiments of the invention, post-bronchodilator absolute FVC is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

In some embodiments, the methods and uses provided herein can increase pre- and post-bronchodilator absolute FVC by at least 3% or at least 4% or at least 5% or at least 6% or at least 7% or at least 8% or at least 9% or at least 10%.

In some embodiments of the invention, pre- and post-bronchodilator absolute FVC is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

Herein, forced expiratory volume in one second (FEV1) is the volume exhaled during the first second of a forced expiratory maneuver started from the level of full inspiration. In different embodiments, the methods provided herein can increase forced expiratory volume in one second (FEV1) in a subject with pulmonary sarcoidosis. An increase can be measured based on the expected FEV1 based on a large patient population, on the FEV1 measured in a control population, or on the individual patient's FEV1 prior to administration of canakinumab (baseline).

In one embodiment, the use or method according to the invention can increase the FEV1, as compared to the patient's baseline FEV1. In some embodiments, the increased FEV1 is pre-bronchodilator FEV1. In some embodiments, the increased FEV1 is post-bronchodilator FEV1. In some embodiments, the increased FEV1 is pre- and post-bronchodilator FEV1. In some embodiments, Spirometry Reversibility Test is performed on a subject with pulmonary sarcoidosis. In one embodiment reversibility (%) is calculated as (FEV1 (post-bronchodilator)−FEV1 (pre-bronchodilator)×100)/FEV1 (pre-bronchodilator).

A “bronchodilator,” as used herein, refers to any drug that widens or dilates the bronchi and bronchioles or air passages of the lungs decreasing resistance in the respiratory airway and increasing airflow to the lungs. For example, bronchodilators include short- and long-acting β2-agonists such as albuterol/salbutamol or others.

In one embodiment of any method or use of the invention pre-bronchodilator FEV1 may improve with the methods and uses according to the present invention in subjects with pulmonary sarcoidosis. Pre-bronchodilator FEV1 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment post-bronchodilator FEV1 may improve with the methods and uses according to the present invention in subjects with pulmonary sarcoidosis. Post-bronchodilator FEV1 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment pre- and post-bronchodilator FEV1 may improve with the methods and uses according to the present invention in subjects with pulmonary sarcoidosis. Pre- and post-bronchodilator FEV1 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

Herein, forced expiratory volume in three seconds (FEV3) is the volume exhaled during the first three seconds of a forced expiratory maneuver started from the level of full inspiration. In different embodiments, the methods and uses provided herein can increase forced expiratory volume in three seconds (FEV3) in a subject with pulmonary sarcoidosis. An increase can be measured based on the expected FEV3 based on a large patient population, on the FEV3 measured in a control population, or on the individual patient's FEV3 prior to administration of canakinumab (baseline).

In one embodiment, the use or method according to the invention can increase the FEV3, as compared to the patient's baseline FEV3. In some embodiments, the increased FEV3 is pre-bronchodilator FEV3. In some embodiments, the increased FEV3 is post-bronchodilator FEV3.

In some embodiments, the increased FEV3 is pre- and post-bronchodilator FEV3.

In one embodiment pre-bronchodilator FEV3 may improve with the methods and uses according to the present invention in subjects with pulmonary sarcoidosis. Pre-bronchodilator FEV3 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment post-bronchodilator FEV3 may improve with the methods and uses according to the present invention in subjects with pulmonary sarcoidosis. Post-bronchodilator FEV3 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment pre- and post-bronchodilator FEV3 may improve with the methods and uses according to the present invention in subjects with pulmonary sarcoidosis. Pre- and post-bronchodilator FEV3 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment. Herein, forced expiratory volume in six seconds (FEV6) is the volume exhaled during the first six seconds of a forced expiratory maneuver started from the level of full inspiration. In different embodiments, the methods and uses provided herein can increase forced expiratory volume in six seconds (FEV6) in a subject with pulmonary sarcoidosis. An increase can be measured based on the expected FEV6 based on a large patient population, on the FEV6 measured in a control population, or on the individual patient's FEV6 prior to administration of canakinumab (baseline).

In one embodiment, the use or method according to the invention can increase the FEV6, as compared to the patient's baseline FEV6. In some embodiments, the increased FEV6 is pre-bronchodilator FEV6. In some embodiments, the increased FEV6 is post-bronchodilator FEV6. In some embodiments, the increased FEV6 is pre- and post-bronchodilator FEV6.

In one embodiment pre-bronchodilator FEV6 may improve with the methods and uses according to the present invention in subjects with pulmonary sarcoidosis. Pre-bronchodilator FEV6 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment post-bronchodilator FEV6 may improve with the methods and uses according to the present invention in subjects with pulmonary sarcoidosis. Post-bronchodilator FEV6 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment pre- and post-bronchodilator FEV6 may improve with the methods and uses according to the present invention in subjects with pulmonary sarcoidosis. Pre- and post-bronchodilator FEV6 is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

Herein, the FEF25-75 is the forced expiratory flow 25%-75% FEV1 forced expiratory volume in 1 second. In some embodiments, the methods and uses described herein can increase the FEF25-75, as compared to the subject's baseline FEF25-75. In some embodiments, the increased FEF25-75 is pre-bronchodilator FEF25-75. In some embodiments, the increased predicted FEF25-75 is post-bronchodilator FEF25-75. In some embodiments, the increased FEF25-75 is pre-bronchodilator FEF25-75 and post-bronchodilator FEF25-75.

In some embodiments of the invention, FEF25-75 pre-bronchodilator is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

In some embodiments of the invention, FEF25-75 post-bronchodilator is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

In some embodiments of the invention, FEF25-75 pre- and post-bronchodilator is increased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

In one embodiment of any method or use of the invention reversibility may improve with the methods and uses according to the present invention in subjects with pulmonary sarcoidosis. Reversibility is increased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment of any use or method of the invention, the subject has improved lung function after at least 12 weeks of treatment, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more compared to before treatment. The improvement of lung function can be determined by spirometry and/or plethysmography.

The term “dyspnea” refers to shortness of breath and may be determined using a variety of assessments. For example, the Modified Medical Research Council (MMRC) dyspnea scale, baseline dyspnea index (BDI), Borg dyspnea score, and/or the oxygen cost diagram (OCD) may be used. For example, the modified Medical Research Council (MMRC) Dyspnea Scale, which is a widely used, rapidly administered, 5-point scale based on degrees of various physical activities that precipitate breathlessness may be used for assessment, wherein “0” on the scale indicates no dyspnea and “5” indicates severe dyspnea.

In one embodiment, use of any of the methods of the invention will reduce the severity of dyspnea in a subject with pulmonary sarcoidosis. On the MMRC Dyspnea Scale, the score will decrease by at least 1 point, at least 2 points, at least 3 points, at least 4 points or 5 points. Dyspnea has improved after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

Parenchymal lung involvement in pulmonary sarcoidosis can be determined, for example, by high-resolution computing tomography (HRCT). Standard plain chest radiographic views are frequently used in the diagnostic and staging processes for sarcoidosis patients. However, the chest radiographic scores (Scadding stages 0-IV) have limited value in predicting severity of pulmonary involvement and are relatively insensitive as a disease marker in therapeutic trials. HRCT (without contrast agent) provides superior resolution of lung morphology when compared to chest radiography or even conventional CT. HRCT can detect parenchymal disease in patients with normal chest radiographs or demonstrate more extensive disease in patients having only focal abnormalities on chest radiographs. Parenchymal disease is characterized by abnormalities observed in the sarcoid parenchyma including, but are not limited to, nodular densities, thickening or irregularity of the bronchovascular bundle, parenchymal opacities, intraparenchymal nodules, patchy areas of ground-glass opacities, irregular linear opacities, alveolar opacities, interstitial thickening, parenchymal consolidation, air cysts, air trapping, septal and nonseptal lines, focal pleural thickening, bronchiectasis, end-stage fibrosis, lymphadenopathy, bilateral hilar lymphadenopathy, mediastinal lymphadenopathy and honeycomb appearance.

In some embodiments of any method according to the invention parenchymal lung involvement has decreased compared to before treatment after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment.

Health-related quality of life in subjects with pulmonary sarcoidosis may be determined using clinical outcomes assessments, for example The King's Sarcoidosis Questionnaire (KSQ) and Functional Assessment of Chronic Illness—Fatigue (FACIT-F). The KSQ is flexible, multi-organ health status measurement consisting of 5 modules: General health status (10 items), Lung (6 items), Medication (3 items), Skin (3 items) and Eye (7 items). The overall score and primary outcome measure is determined by combining modules, with the individual modules identifying the health domains affected. Scores range from 0 to 100, with the higher score indicating better health status.

Fatigue is a significant problem for more than one-half of sarcoidosis patients and a major cause of their impaired quality of life. FACIT-F self-report fatigue questionnaire has shown a valid and reliable fatigue measure in a broad and diverse range of diseases including sarcoidosis. The maximum score of FACIT is 52, with higher scores indicating more fatigue.

In one embodiment of any use or method of the invention the quality of life is assessed, for example, by KSQ. In one embodiment the KSQ score of the subjects with pulmonary sarcoidosis increases after at least 12 weeks of treatment or after at least 24 weeks of treatment.

In one embodiment of any use or method of the invention fatigue is decreased as determined, for example, by FACIT-F. In one embodiment fatigue of the subjects with pulmonary sarcoidosis as assessed by the FACIT-F score decreases after at least 12 weeks of treatment or after at least 24 weeks of treatment.

Pulmonary function may be assessed by determining the diffusing capacity of the lung (DL), which measures the transfer of gas from air in the lung to erythrocytes in lung blood vessels.

In one embodiment, diffusing capacity for carbon monoxide (DL_{CO}) is determined according to ATS guidelines (Macintyre et al. 2005). Measurements may include DL_{CO} and alveolar volume (VA). DL_{CO} may be determined by measuring the uptake of carbon monoxide from the lung over a breath-holding period. VA represents an estimate of lung gas volume into which CO is distributed and then transferred across the alveolar capillary membrane making it critical in the measurement of DL_{CO}. VA is typically measured simultaneously with CO uptake by calculating the dilution of an inert tracer gas (e.g. argon, methane or helium). In normal subjects, the sum of VA and VD (dead space volume) closely matches the total lung capacity (TLC) determined by plethysmography. However, in cases of poor gas mixing in patients with obstructed airways, tracer gas dilution is markedly reduced leading to VA values that are significantly less than those expected based on actual total thoracic gas volumes.

In one embodiment of any method or use of the invention, the subject has improved single breath DL_{CO} after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

Absolute lung volumes can be measured, for example, with plethysmography. Parameters of absolute lung volumes include, but are not limited to, Functional Residual Capacity (FRC), Inspiratory Capacity (IC), Total Lung Capacity (TLC), and Residual Volume (RV). Plethysmography evaluations should follow the recommendations of the ATS/ERS Task force: Standardization of the measurement of lung volumes (Wanger et al. 2005).

Functional Residual Capacity (FRC) is the volume of gas present in the lung at end-expiration during tidal breathing. Inspiratory Capacity (IC) is the maximum volume of gas that can be inspired from FRC. Total Lung Capacity (TLC) refers to the volume of gas in the lungs after maximal inspiration, or the sum of all volume compartments. Residual Volume (RV) refers to the volume of gas remaining in the lung after maximal exhalation (regardless of the lung volume at which exhalation was started).

In one embodiment of any method or use of the invention, the subject has improved Functional Residual Capacity (FRC) after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment of any method or use of the invention, the subject has improved Inspiratory Capacity (IC) after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment of any method or use of the invention, the subject has improved Total Lung Capacity (TLC) after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

In one embodiment of any method or use of the invention, the subject has improved Residual Volume (RV) after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

[F-18]FDG-PET may detect increased inflammation-associated metabolic activity in sarcoidosis. The glucose analogue fluorodeoxyglucose (FDG) is labelled with a positron emitting fluorine-18 and [F-18]FDG can be used in positron emission tomography (PET) to visualise metabolic activity of inflammation. Active granulomas appear to have a high affinity for FDG, reflecting the high sensitivity of [F-18]FDG-PET imaging. Assessment of the metabolic activity of sarcoidosis [F-18]FDG-PET can be determined by maximum standardized uptake value (SUVₘₐₓ), for example, in focal nodal uptake regions (mediastinal, hilar), focal regions of uptake in lung parenchyma and/or extra-thoracic focal uptake regions

In some embodiments of any method or use of the invention the maximum standardized uptake value (SUVₘₐₓ) ([F-18]-FDG-PET) has decreased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more of treatment compared to before treatment.

Other parameters captured by [F-18]FDG-PET imaging may include, but are not limited to, mean standardized uptake value (SUVₘₑₐₙ), peak standardized uptake value (SUVₚₑₐₖ) and volume of the lesions.

In some embodiments of any method or use of the invention the SUVₘₑₐₙ captured by [F-18]FDG-PET has decreased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more from first administration compared to before treatment.

In some embodiments of any method or use of the invention the SUVₚₑₐₖ captured by [F-18]FDG-PET has decreased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more from first administration compared to before treatment.

In some embodiments of any method or use of the invention the volume of the lesions captured by [F-18]FDG-PET has decreased after at least 4 weeks, after at least 8 weeks, after at least 12 weeks, after at least 16 weeks, after at least 20 weeks, after at least 24 weeks, after at least 28 weeks, after at least 32 weeks, after at least 36 weeks, after at least 40 weeks, after at least 44 weeks, after at least 48 weeks, after at least 52 weeks or more from first administration compared to before treatment.

In other embodiments of any method according to the invention, biomarkers may include, but are not limited to: serum levels of soluble IL-2 receptor (sIL-2R), interleukin-18 (IL-18), interleukin-18 binding protein (IL-18 bp), serum angiotensin converting enzyme (ACE), serum amyloid A protein, chitotriosidase (ChT), circulating fibrocytes, bronchoalveolar lavage (BAL) total cell count and BAL neutrophil cell count and Th-1 related biomarkers. Biomarkers can be used for assessing the response to canakinumab compared to between baseline (before administration) and after administration.

The 6MWT as mentioned herein refers to the standard physical exercise test performed in accordance with current clinical practice, e.g. as defined in the current practical guidelines provided by medical societies, e.g. the American Thoratic Society, e.g. as described in ATS Statement: Guidelines for the Six-Minute Walk Test, Am J Respir Crit Care Med Vol 166. pp 111-117, 2002. Preferably, the 6MWT is performed in accordance to said ATS Statement of 2002.

In some embodiments, the subject's ability to walk for 6 min will improve after treatment with the methods and uses according to the present invention.

In some embodiments, the subject's ability for physical activity will improve, determined by the 6 minute walk test (6MWT), in respect to at least one of the following:
a walk distance-in-6 minutes increase, preferably by at least 20 m, more preferably at least 50 m or by at least 5%, preferably at least 10%, more preferably at least 15%, even more preferably at least 20%,
dyspnea-free walk distance increase of at least 5%, preferably at least 10%, more preferably at least 15%, even more preferably at least 20%,
a maximum walk distance increase by at least 5%, preferably at least 10%, more preferably at least 15%, even more preferably at least 20%,

after at least 52, 36, 24, or 12 weeks of treatment compared to before treatment (baseline). As used herein, the terms “treat”, “treatment” and “treating” refer to the reduction or amelioration of the progression, severity and/or duration of pulmonary sarcoidosis, or the amelioration of one or more symptoms, suitably of one or more discernible symptoms, of pulmonary sarcoidosis resulting from the administration of canakinumab. In specific embodiments, the terms “treat”, “treatment” and “treating” refer to the amelioration of at least one measurable physical parameter of pulmonary sarcoidosis, wherein the physical parameter is not necessarily discernible by the patient.

In one embodiment of any method or use of the invention, canakinumab is administered every 2 weeks, monthly, bimonthly (every 2 months), quarterly (every 3 months), half-yearly, every 16 weeks, every 4 months, every 5 months, or every 6 months or every 4 weeks, every 6 weeks, every 8 weeks, every 12 weeks, every 16 weeks, every 20 weeks, every 24 weeks from the first administration. In one embodiment, canakinumab is administered monthly.

One embodiment of any method or use of the invention further comprises administering the patient an additional dose of about 25 mg to about 300 mg of canakinumab at week 2, week 4, week 6, week 8, week 12, week 16, week 20 or week 24 or 1 month, 2 months, 3 months, 4 months, 5 months or 6 months from first administration.

One embodiment of any method or use of the invention comprises administering about 25, 75, 80, 100, 125, 150, 175, 200, 225, 250, 275, 300 mg or any combination thereof of canakinumab. In other embodiments of the administration regimens described above, a dose of about 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 mg or any combination thereof of canakinumab can be administered. In some embodiments of any of the methods and uses described above, canakinumab is administered parenterally, for example subcutaneously or intravenously. Preferably, canakinumab is administered subcutaneously.

When administered parenterally, e.g. subcutaneously or intravenously, canakinumab can be administered in a reconstituted formulation comprising: 10-200 mg/ml canakinumab, sucrose, histidine and polysorbate 80, wherein the pH of the formulation is 6.1-6.9, preferably 6.5 or 10-200 mg/ml canakinumab, 270 mM sucrose, 30 mM histidine and 0.06% polysorbate 80, wherein the pH of the formulation is 6.5. When administered parenterally, e.g. subcutaneously or intravenously, canakinumab can be administered in a liquid formulation comprising: 10-200 mg/ml canakinumab, mannitol, histidine and polysorbate 80 (or polysorbate 20), wherein the pH of the formulation is 6.1-6.9, preferably 6.5 or 10-200 mg/ml of canakinumab, 270 mM mannitol, 20 mM histidine and 0.04% polysorbate 80 (or polysorbate 20), wherein the pH of the formulation is 6.1-6.9, preferably 6.5. When administered subcutaneously, canakinumab can be administered to the patient in a liquid form or lyophilized form for reconstitution. Preferably such liquid formulation is contained in a prefilled syringe that can be stored for at least 2 years. In one embodiment said prefilled syringe can be contained in an autoinjector. Such autoinjector makes it possible for the patient to self-administer the liquid formulation subcutaneously in an easy manner.

When administered subcutaneously, canakinumab can be administered to the patient in a liquid form or lyophilized form for reconstitution contained in a prefilled syringe. In one embodiment, the prefilled syringe is contained in an autoinjector.

In another embodiment of any method or use of the invention, said patient may concomitantly receive a glucocorticoid such as methylprednisolone or prednisone and/or an immunosuppressive agent such as methotrexate, azathioprine, leflunomide, hydroxychloroquine or mycophenolate.

### General:

All patents, published patent applications, publications, references and other material referred to herein are incorporated by reference herein in their entirety.

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 10% from the stated amount: this usage of “about” recognizes that the precise amount in a given dosage form may differ slightly from an intended amount for various reasons without materially affecting the in vivo effect of the administered compound.

The terms “comprising” and “including” are used herein in their open-ended and non-limiting sense unless otherwise noted.

As used herein, the term “comprising” encompasses “including” as well as “consisting,” e.g. a composition “comprising” X may consist exclusively of X or may include something additional, e.g., X+Y.

As used herein, the term “administering” in relation to a compound, e.g., an IL-1β binding antibody, e.g. canakinumab, or standard of care agent, is used to refer to delivery of that compound by any route of delivery.

As used herein, the word “substantially” does not exclude “completely,” e.g. a composition which is “substantially free” from Y may be completely free from Y. Where necessary, the word “substantially” may be omitted from the definition of the invention.

As used herein, the term “patient” and “subject” includes any human or nonhuman animal and can be used interchangeably. The term “nonhuman animal” includes all vertebrates, e.g. mammals and non-mammals, such as nonhuman primates, sheep, dogs, cats, horses, cows, chickens, amphibians, reptiles, etc.
As used herein, the term “baseline” denotes a given parameter or the state of the patient before administration of canakinumab.

Without intending to limit the scope of the invention in any way, it is further described by way of illustration of the following example.

### Example

A Double-Blinded, Randomized, Placebo-Controlled, Parallel-Group, Non-Confirmatory Study to Evaluate the Efficacy, Safety and Tolerability of Canakinumab in Patients with Pulmonary Sarcoidosis

The study will randomize patients with evidence of parenchymal involvement and histologically proven, chronic pulmonary sarcoidosis of ≥1 year duration with persisting activity at baseline despite background therapy as determined by clinical history, radiologic evidence (e.g., HRCT), spirometry and MMRC dyspnea scale assessments. Randomization will be stratified by positive [F-18]FDG-PET/CT parenchymal tracer uptake (yes/no). [F-18]FDG-PET/CT imaging must also have been done without changes in treatment during the subsequent period leading up to study randomization. Acceptable background therapy includes a stable regimen of prednisone or equivalent ≤15 mg/day and/or no more than one immune suppressive agent (e.g. methotrexate, azathioprine, leflunomide or hydroxychloroquine).

Subjects who meet the eligibility criteria at screening will undergo evaluation of full baseline clinical and biomarker assessments prior to injection. Baseline assessments including safety laboratory evaluations and pulmonary function tests will not be available prior to dosing and for those measures eligibility criteria will be determined based on the screening results. Enrolled subjects will be randomized at a 1:1 ratio to receive treatment with either ACZ885 (canakinumab) or placebo. On Day 1, every four week (28 days) s.c. dosing with ACZ885 will begin at 300 mg. Patients in the placebo treatment arm will be injected in a like manner with placebo. All patients will return to the study center for safety and pharmacokinetic (PK) checks on an every four week basis at which time they will receive either study treatments depending on treatment arm. Additionally, patients will undergo clinical assessments that include pulmonary function tests with lung volumes, DL_{CO}, 6MWT, and clinical outcome assessments.

At week 12, a second [F-18]FDG-PET/CT will be obtained. In addition, functional clinical measures and biomarker assessment will take place at this time point. The final dosing will take place on week 20, followed by a visit on week 24 that will include assessments for clinical outcomes and biomarkers. Also included at the week 24 visit is the second HRCT assessment. Patients return for the end of study (EOS) visit at week 32.

### Objectives:

To compare the effect of ACZ885 versus placebo on the clinical disease activity of sarcoidosis patients as measured by the change from baseline in the percent predicted forced vital capacity (FVC) at week 24
To determine the effect of ACZ885 on decreasing the maximum standardized uptake value (SUVₘₐₓ) [F-18]FDG-PET in nodules (nodular uptake regions) after 12 weeks of treatment, compared to placebo
To determine the effect of ACZ885 versus placebo on parameters of pulmonary function testing (e.g. absolute FVC, FEV1, FEV1/FVC, FEV3, FEV6, FEF25-75, FEV3/FVC, 1-(FEV3/FVC), TLC, RV, RV/TLC, DL_{CO} and postbronchodilator FEV1/reversibility) in patients with sarcoidosis at 24 weeks compared to baseline
To determine the effect of ACZ885 versus placebo on HRCT of patients with sarcoidosis at 24 weeks compared to initial HRCT scan as measured by side-by-side comparison by blinded reviewers and HRCT scoring
To determine the effect of ACZ885 versus placebo on the 6-minute walk test (6MWT) distance of patients with sarcoidosis at 12 and 24 weeks compared to baseline
To determine the effect of ACZ885 on additional [F-18]FDG-PET outcomes (e.g. SUVₘₑₐₙ, SUVpeak and volume of the lesions) after 12 weeks compared to placebo

### High Resolution Computed Tomography

High resolution computed tomography (HRCT) is used in this study for both screening and for clinical outcome measurements. Standard plain chest radiographic views are frequently used in the diagnostic and staging processes for sarcoidosis patients. However, the chest radiographic scores (Scadding stages 0-IV) have limited value in predicting severity of pulmonary involvement and are relatively insensitive as a disease marker in therapeutic trials. HRCT (without contrast agent) provides superior resolution of lung morphology when compared to chest radiography or even conventional CT. HRCT can detect parenchymal disease in patients with normal chest radiographs or demonstrate more extensive disease in patients having only focal abnormalities on chest radiographs (Batra 1993, Drent et al. 2003).

### [F-18]FDG-PET/CT Imaging

F-18]FDG-PET/CT imaging to provide early evidence for effective decrease in IL-1β-driven inflammation on ACZ885 treatment. [F-18]FDG-PET/CT detects increased inflammation-associated metabolic activity in sarcoidosis with sensitivity of 90-100%, and decreases in [F-18]FDG-PET/CT at 24 weeks compared to baseline have been correlated with improvements in FVC over this time period (Keijsers et al. 2008, Milman et al. 2012, Adams et al. 2014).

### Pulmonary Function Tests

Pulmonary function tests include spirometry (forced vital capacity (FVC): absolute and FVC % (forced vital capacity, expressed as a percentage of the normal expected value) and forced expiratory volume in one second (FEV1), FEF25-75, FEV1/FVC, FEV3/FVC, 1-(FEV3/FVC), FEV6, plethysmography (Functional Residual Capacity (FRC), Inspiratory Capacity (IC), Total Lung Capacity (TLC), Residual Volume (RV), and RV/TLC) and diffusion capacity for carbon monoxide (DL_{CO}) and alveolar volume (VA) to allow further characterization of the patients' response to treatment.

### 6 Minute Walk Test (6MWT)

The 6MWT (including distance walked in meters, oxygen saturation in %, heart rate in beats per minute (bpm) and Borg Questionnaire score) is a practical and simple assessment of functional capacity, reflective of activities of daily living (Enright 2003) that has been increasingly applied to assess various lung diseases, including interstitial lung diseases other than sarcoidosis where it has proved useful for both predicting mortality and monitoring response to therapy.

### Clinical Outcome Assessments (COAs)

Health-related quality of life and health status in interstitial lung diseases are important parameters of disease activity and prognosis. Both disease symptoms and treatment side effects can impact on patients' quality of life. Health-related quality of life is determined through clinical outcomes assessments (COAs), e.g. The King's Sarcoidosis Questionnaire (KSQ) and Functional Assessment of Chronic Illness—Fatigue (FACIT-F)

### Study Design

The study will randomize approximately 38 patients (targeting 30 completers) with evidence of parenchymal involvement and histologically proven, chronic pulmonary sarcoidosis of ≥1 year duration with persisting activity at baseline despite background therapy as determined by clinical history, radiologic evidence (e.g. HRCT, MM or chest x-ray), spirometry and MMRC dyspnea scale assessments.

For each subject, there will be a maximum 40-day screening period. Screening is valid for 40 days from the time of the first screening assessment. [F-18]FDG-PET/CT scans obtained locally at the study site after at least 8 weeks of dosing will be acceptable for baseline assessment prior to receiving the first injection of ACZ885. However, any prior [F-18]FDG-PET/CT imaging must also have been done without changes in treatment during the subsequent period leading up to study randomization.

Subjects who meet the eligibility criteria at screening will undergo evaluation of full baseline clinical and biomarker assessments prior to injection. Baseline assessments including safety laboratory evaluations and pulmonary function tests will not be available prior to dosing and for those measures eligibility criteria will be determined based on the screening results. Enrolled subjects will be randomized at a 1:1 ratio to receive treatment with either ACZ885 or placebo. On Days 1, 29, 57, 85, 113 and 141 patients will be administered s.c. dosing with ACZ885 at 300 mg or corresponding placebo treatment. All patients will return to the study center for safety and pharmacokinetic (PK) checks on an every four week basis at which time they will receive either study treatments depending on treatment arm. Additionally, patients will undergo clinical assessments that include pulmonary function tests with lung volumes, DLCO, 6MWT, and clinical outcome assessments as on days 1, 29, 57, 85, 113, 141 and 169.

At week 12, a second [F-18]FDG-PET/CT will be obtained. In addition, functional clinical measures and biomarker assessment will take place at this time point. The final dosing will take place on week 20, followed by a visit on week 24 that will include assessments for clinical outcomes and biomarkers. Also included at the week 24 visit is the second HRCT assessment. Patients return for the end of study (EOS) visit at week 32.

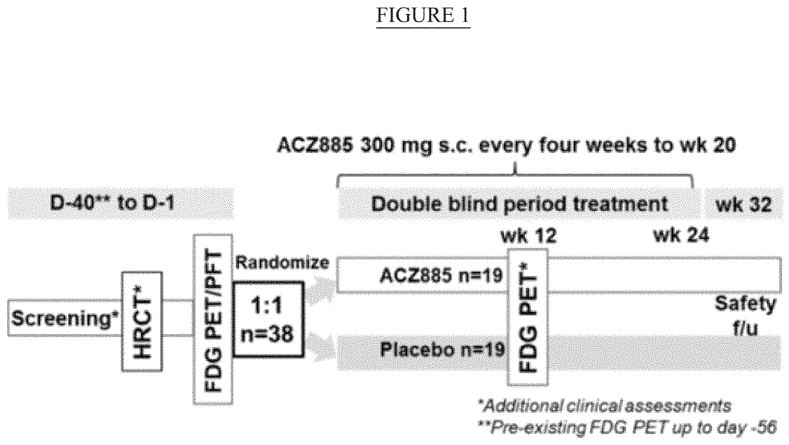

For graphic of study design see FIG. 1.

### Inclusion Criteria

Pulmonary sarcoidosis patients eligible for inclusion in this study must fulfill all of the following criteria:
1. Written informed consent must be obtained before any assessment is performed.
2. Male and female subjects ages 18 to 80 years of age (both inclusive).
3. Subjects must weigh at least 50 kg to participate in the study.
4. Able to communicate well with the investigator and to understand and comply with the requirements of the study.
5. Disease duration of ≥1 year
6. Clinically active disease demonstrated either by a biopsy (any organ) or by bronchoalveolar lavage (lymphocytosis >15%, CD4+/CD8+ ratio >3.5, CD103+CD4+/CD4+ ratio <0.2). Patients must also have all of the following criteria:
   MMRC dyspnea scale≥1
   Threshold FVC 50-90% of predicted
   Evidence of parenchymal lung involvement by HRCT at screening or by historical radiological evidence (e.g. CT, MM or x-ray)

### REFERENCES

Adams, H., Keijsers, R. G., Korenromp, I. H. and Grutters, J. C. (2014) ‘FDG PET for gauging of sarcoid disease activity’, Semin Respir Crit Care Med, 35(3), 352-61.
Batra, P. (1993) ‘Role of high-resolution CT in the diagnosis and evaluation of pulmonary sarcoidosis’, Sarcoidosis, 10(2), 95-7.
Baughman, R. P. and Lower, E. E. (2011) ‘Who dies from sarcoidosis and why?’, Am J Respir Crit Care Med, 183(11), 1446-7.
Baughman, R. P. and Nunes, H. (2012) ‘Therapy for sarcoidosis: evidence-based recommendations’, Expert Rev Clin Immunol, 8(1), 95-103.
Chen, E. S. and Moller, D. R. (2011) ‘Sarcoidosis—scientific progress and clinical challenges’, Nat Rev Rheumatol, 7(8), 457-67.
Devergne, O., Emilie, D., Peuchmaur, M., Crevon, M. C., D'Agay, M. F. and Galanaud, P. (1992) ‘Production of cytokines in sarcoid lymph nodes: preferential expression of interleukin-1 beta and interferon-gamma genes’, Hum Pathol, 23(3), 317-23.
Drent, M., De Vries, J., Lenters, M., Lamers, R. J., Rothkranz-Kos, S., Wouters, E. F., van Dieijen-Visser, M. P. and Verschakelen, J. A. (2003) ‘Sarcoidosis: assessment of disease severity using HRCT’, Eur Radiol, 13(11), 2462-71.
Enright, P. L. (2003) ‘The six-minute walk test’, Respir Care, 48(8), 783-5.
Hunninghake, G. W. (1984) ‘Release of interleukin-1 by alveolar macrophages of patients with active pulmonary sarcoidosis’, Am Rev Respir Dis, 129(4), 569-72.
Kasahara, K., Kobayashi, K., Shikama, Y., Yoneya, I., Kaga, S., Hashimoto, M., Odagiri, T., Soejima, K., Ide, H., Takahashi, T. and et al. (1989) ‘The role of monokines in granuloma formation in mice: the ability of interleukin 1 and tumor necrosis factor-alpha to induce lung granulomas’, Clin Immunol Immunopathol, 51(3), 419-25.
Keijsers, R. G., Verzijlbergen, J. F., van Diepen, D. M., van den Bosch, J. M. and Grutters, J. C. (2008) ‘18F-FDG PET in sarcoidosis: an observational study in 12 patients treated with infliximab’, Sarcoidosis Vasc Diffuse Lung Dis, 25(2), 143-9.
Macintyre, N., Crapo, R. O., Viegi, G., Johnson, D. C., van der Grinten, C. P., Brusasco, V., Burgos, F., Casaburi, R., Coates, A., Enright, P., Gustafsson, P., Hankinson, J., Jensen, R., McKay, R., Miller, M. R., Navajas, D., Pedersen, O. F., Pellegrino, R. and Wanger, J. (2005) ‘Standardisation of the single-breath determination of carbon monoxide uptake in the lung’, Eur Respir J, 26(4), 720-35.
Milman, N., Graudal, N., Loft, A., Mortensen, J., Larsen, J. and Baslund, B. (2012) ‘Effect of the TNF-alpha inhibitor adalimumab in patients with recalcitrant sarcoidosis: a prospective observational study using FDG-PET’, Clin Respir J, 6(4), 238-47.
Paramothayan, S. and Lasserson, T. (2008) ‘Treatments for pulmonary sarcoidosis’, Respir Med, 102(1), 1-9.
Terao, I., Hashimoto, S. and Horie, T. (1993) ‘Effect of GM-CSF on TNF-alpha and IL-1-beta production by alveolar macrophages and peripheral blood monocytes from patients with sarcoidosis’, Int Arch Allergy Immunol, 102(3), 242-8.
Wanger, J., Clausen, J. L., Coates, A., Pedersen, O. F., Brusasco, V., Burgos, F., Casaburi, R., Crapo, R., Enright, P., van der Grinten, C. P., Gustafsson, P., Hankinson, J., Jensen, R., Johnson, D., Macintyre, N., McKay, R., Miller, M. R., Navajas, D., Pellegrino, R. and Viegi, G. (2005) ‘Standardisation of the measurement of lung volumes’, Eur Respir J, 26(3), 511-22.

1. A method of treating or alleviating the symptoms of pulmonary sarcoidosis in a subject, comprising administering about 25 mg to about 300 mg of canakinumab.

2. The method according to claim 1, wherein the subject is exhibiting at least one of the following conditions before treatment:
a. Reduced lung function
b. Dyspnea of at least 1 on the Modified Medical Research Council (MMRC) Dyspnea scale
c. Abnormalities in the lung parenchyma

3. The method according to claim 1, wherein the subject has predicted forced vital capacity (% FVC) of ≤80%, ≤85%, or ≤90% before treatment.

4-5. (canceled)

6. The method according to claim 1, wherein the subject has greater than 3% improvement in predicted forced vital capacity (FVC) after at least 24 weeks of treatment compared to before treatment.

7. The method according to claim 1, wherein the subject has improved lung function as determined by spirometry, plethysmography, and/or diffusing capacity of carbon monoxide (DLCO) after at least 24 weeks of treatment compared to before treatment.

8-9. (canceled)

10. The method according to claim 1, wherein the subject has improved ability for physical activity, determined by the 6 minute walk test (6MWT), of at least one of the following:
a walk distance-in-6 minutes increase,
dyspnea-free walk distance increase,
a maximum walk distance increase,
after at least 12 weeks of treatment compared to before treatment.

11. The method according to claim 1, wherein the subject has improved ability for physical activity, determined by the 6 minute walk test (6MWT), of at least one of the following:
a walk distance-in-6 minutes increase,
dyspnea-free walk distance increase,
a maximum walk distance increase,
after at least 24 weeks of treatment compared to before treatment.

12. The method according to claim 1, wherein the subject has decreased parenchymal abnormalities after at least 24 weeks of treatment compared to before treatment.

13. The method according to claim 1, wherein the parenchymal abnormalities are detected by high-resolution computing tomography (HRCT).

14. The method according to claim 1, wherein the subject experiences improvements on the MMRC scale of dyspnea of at least 1 point after at least 24 weeks of treatment compared to before treatment.

15. The method according to claim 1, wherein canakinumab is administered twice a month, monthly, quarterly, every 2 months, every 3 months, every 4 months, every 5 months, or every 6 months; or every 2 weeks, every 4 weeks, every 6 weeks, every 8 weeks, every 12 weeks, every 16 weeks, every 20 weeks, or every 24 weeks.

16-17. (canceled)

18. The method according to claim 1, wherein the method comprises administering about 25 mg, about 50 mg, about 75 mg, about 80 mg, about 100 mg, about 125 mg, about 150 mg, about 175 mg, about 200 mg, about 225 mg, about 250 mg, about 275 mg, about 300 mg, or any combination thereof of canakinumab.

19. The method according to claim 1, wherein said method comprises administering about 50 mg, about 80 mg, about 150 mg, about 200 mg, or about 300 mg of canakinumab.

20-23. (canceled)

24. The method according to claim 1, further comprising administering the patient an additional dose of about 25 mg to about 300 mg of canakinumab at about week 2, about week 4, or about week 6; or about 2 months, or about three months, or about four months, or about five months, or about six months from first administration.

25. The method according to claim 24, wherein the additional dose is about 50 mg, about 80 mg, or about 150 mg or about 300 mg of canakinumab.

26. The method according to claim 1, wherein canakinumab is administered subcutaneously.

27. (canceled)

28. The method according to claim 26, wherein canakinumab is administered in a liquid formulation comprising canakinumab at a concentration of 10-200 mg/ml.

29. The method according to claim 1, wherein canakinumab is administered to the subject in a liquid form contained in a prefilled syringe.

30. The method according to claim 29, wherein the prefilled syringe is contained in an autoinjector.

31. The method according to claim 1, wherein the subject is concomitantly receiving a glucocorticoid and/or an immunosuppressive agent such as methotrexate, azathioprine, leflunomide, hydroxychloroquine or mycophenolate.

32-63. (canceled)
